(19) United States
(12) Plant Patent Application Publication
(10) Pub. No.: US 2002/0157160 P1
(43) Pub. Date: Oct. 24, 2002

(54) PETUNIA PLANT NAMED 'DANCASPURSPARK'

(76) Inventor: Gabriel Danziger, Nir-Zvi (IL)

Correspondence Address: FOLEY & LARDNER, 3000 K Street, N.W., Suite 500, Washington, DC, 20007-5109, US

(21) Appl. No.: 09/838,230

(22) Filed: Apr. 20, 2001

(51) Int. Cl. A01H 5/00

(52) U.S. Cl. PLT/356

(57) ABSTRACT

A new and distinct cultivar of petunia plant named ‘Dancasoft’, as described and illustrated, and particularly characterized by the combined features of pale purple flower color with vibrant purple veins, single flower type, early year round flowering time, medium flower size, spreading, well branched, vigorous growth habit, very floriferous, long bloom season, good weather tolerance, suitable for hanging baskets and mass covering.

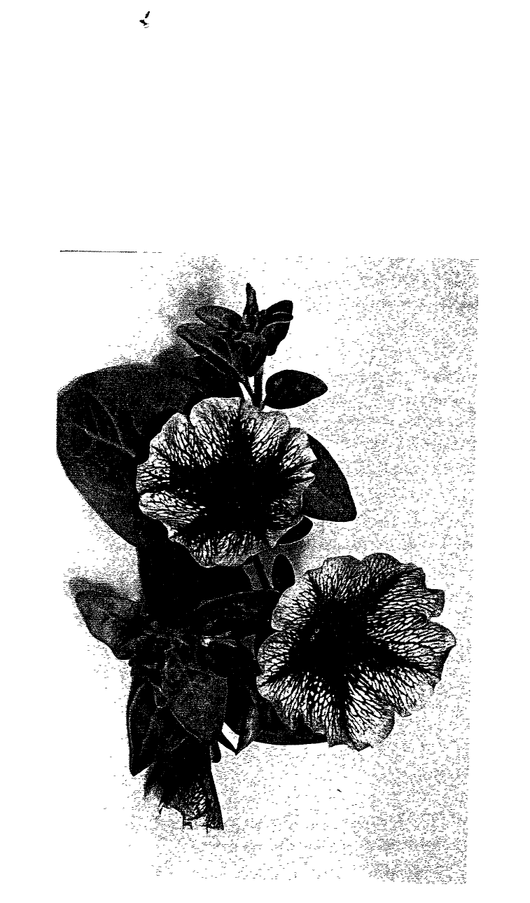

### BACKGROUND OF THE INVENTION

The present invention comprises a new and distinct cultivar of petunia, known by the cultivar name ‘Dancaspurspark’. ‘Dancaspurspark’ was originated from a hybridization made by the inventor in a controlled breeding program in Mishmar Hashiva, Israel in 1999. The female was ‘Pink Spark’. The male parent of was ‘Dancaspurspark’ was ‘CV. 4021’.

‘Dancaspurspark’ was discovered and selected as one flowering plant within the progeny of the stated cross by or under the supervision of the inventor Gabriel Danziger in May, 1999, in a controlled environment in Mishmar Hashiva, Israel.

The first act of asexual reproduction of ‘Dancaspurspark’, was accomplished when vegetative cuttings were taken from the initial selection in June 1999 in a controlled environment in Mishmar Hashiva, Israel by a technician working under formulations established and supervised by the inventor. Horticultural examination of selected units initiated September, 1999, has demonstrated that the combination of characteristics as herein disclosed for ‘Dancaspurspark’ are firmly fixed and are retained through successive generations of asexual reproduction.

‘Dancaspurspark’ has not been observed under all possible environmental conditions. The phenotype may vary somewhat with variations in environment such as temperature, light intensity and day length, however, without any variations in genotype. The following observations, measurements and comparisons describe the plants grown in Mishmar Hashiva, Israel, under greenhouse conditions which approximate those generally used in commercial practice.

### BRIEF DESCRIPTION OF THE INVENTION

The following traits have been repeatedly observed and are determined to be basic characteristics of ‘Dancaspurspark’ which in combination distinguish this petunia as a new and distinct cultivar:

1. pale purple flower color and vibrant purple veins;

2. single flower type;

3. early year round flowering time;

4. medium flower size;

5. spreading, well branched, vigorous growth habit, very floriferous, long bloom season;

6. good weather tolerance; and

7. suitable for hanging baskets and mass covering.

Of the many commercial cultivars known to the present Inventor (s), the most similar in comparison to ‘Dancaspurspark’ is ‘Pink Spark’ by Danziger. In comparison to Pink Spark, ‘Dancaspurspark’ has bigger flowers, is more floriferous, has better branching, is more trailing, has earlier flowering, and with darker shades and more brilliant flower color.

### BRIEF DESCRIPTION OF THE DRAWING

The accompanying photographic drawing(s) show typical Flower and foliage characteristics of ‘Dancaspurspark’, with the color being as true as possible with illustrations of this type.

In the following description color references are made to The Royal Horticultural Society Colour Chart. The color values were determined between 10:00 a.m. and 12:00 p.m. in October, 2000, under full sunlight at Mishmar Hashiva, Israel.

Classification:

Botanical.—A hybrid of petunia Petunia hybrida

Commercial.—Petunia cultivar Trailing Petunia

Asexual Reproduction.—By vegetative propagation

Plant:

Growth Habit.—Trailing vigorously

Plant Height.—6-8 inches

Spreading Area.—30 inches

Blooming Period.—All year found in moderate climate

Stem:

Thickness.—4 mm

Color.—Green RHS 143 B

Pubescence.—Yes

Branching.—Freely

Internode Length.—10-20 mm

Leaf:

Arrangement on stem.—Alternate

Shape.—Elliptic

Size.—Length: 70 mm Width: 35 mm

Thickness.—0.2 mm

Color.—Green RHS 137 C

Markings.—None

Pubescence.—Yes

Flower:

Orientation at opening.—Upright

Type.—Single

Shape.—Regular, rounded. corolla united with 5 lobes and a long straight tube

Diameter.—55 mm

Color.—Upper and under surface color: Violet RHS 88 C Pattern color of veins: Red-Purple RHS 71 A

Peduncle.—Length: 10-15 mm Color: Yellow-green RHS 144 B

Rreproductive Organs:

Pistil.—1 Color: Violet 86 C

Stamens.—5

Anther Color.—Violet-blue RHS 92 A

Filament Color.—Violet-blue RHS 92 D

Weather Resistance: Relatively high rain, heat, and drought resistance

Disease/Pest Resistance: Unknown

I claim:

1. A new and distinct cultivar of petunia plant named ‘Dancaspurspark’, as described and illustrated.
